(19) United States
(12) Patent Application Publication
(10) Pub. No.: US 2024/0160710 A1
(43) Pub. Date: May 16, 2024

(54) REPORTING SYSTEM, REPORTING METHOD, AND RECORDING MEDIUM

(71) Applicants: NEC Corporation, Minato-ku, Tokyo (JP); NEC Platforms, Ltd., Kawasaki-shi, Kanagawa (JP)

(72) Inventor: Ryoji Miyahara, Kanagawa (JP)

(73) Assignees: NEC Corporation, Minato-ku, Tokyo (JP); NEC Platforms, Ltd., Kawasaki-shi, Kanagawa (JP)

(21) Appl. No.: 18/282,750

(22) PCT Filed: Mar. 18, 2021
(86) PCT No.: PCT/JP2021/011024

§ 371 Date: Sep. 18, 2023

(51) Int. Cl. G06F 21/32; H04R 1/32

(52) U.S. Cl. CPC G06F 21/32; H04R 1/323

(57) ABSTRACT

A reporting system determines whether an earphone is worn in one ear of a user, reports instruction information instructing the user to wear the earphone when it is determined that the earphone is not worn, and authenticates the user by otoacoustic authentication when it is determined that the earphone is worn in one ear after the instruction information is reported. The otoacoustic authentication is an authentication method that sends out a test sound in the direction of the ear canal of the one ear or other ear of the user, and authenticates the user based on the reflected sound to the test sound.

### TECHNICAL FIELD

This disclosure relates to a reporting system, a reporting method, and a recording medium.

### BACKGROUND ART

Patent Document 1 discloses an invention for determining whether a headset or the like is worn on an ear based on a wear detection signal such as a test signal and a reflection signal.

### PRIOR ART DOCUMENTS

### Patent Documents

Patent Document 1: Japanese Unexamined Patent Application, First Publication No. 2009-232423

### SUMMARY OF THE INVENTION

### Problems to be Solved by the invention

The invention described in Patent Document 1 could only detect whether or not a headset or other device was being worn.

In view of the above circumstances, it is an example object of this invention to provide a reporting system, a reporting method, and a recording medium that solve the above problem.

### Means for Solving the Problem

One example aspect of this disclosure is a reporting system including a determination means that determines whether a first earphone is worn in one ear of a user, a reporting means that receives the request and reports instruction information instructing the user to wear the first earphone when the determination means has determined that the earphone is not worn, and an authentication means that authenticates the user by otoacoustic authentication when the determination means has determined that the first earphone is worn in the one ear after the reporting means has reported the instruction information, wherein the otoacoustic authentication is an authentication method that sends out a test sound in the direction of the ear canal of the one ear or other ear of the user, and authenticates the user based on the reflected sound to the test sound.

### BRIEF DESCRIPTION OF THE DRAWINGS

FIG. 1 is a diagram that shows a configuration example of the reporting system S in the first example embodiment.

FIG. 2 is a block diagram showing the hardware configuration of the information communication device 1 in the first example embodiment.

FIG. 3 is a block diagram showing the hardware configuration of earphone 2 in the first example embodiment.

FIG. 4 is a block diagram showing the hardware configuration of the earphone control device 20 in the first example embodiment.

FIG. 5 is a functional block diagram of the information communication device 1 in the first example embodiment.

FIG. 6 is a functional block diagram of the earphone control device 20 in the first example embodiment.

FIG. 7 is a flowchart showing the process flow of the reporting system S in the first example embodiment.

FIG. 8 is a diagram that shows an example of the reporting information in the first example embodiment.

FIG. 9 is a flowchart showing the process flow of the reporting system S in the second example embodiment.

FIG. 10 is block diagram showing the hardware configuration of earphone 2B in the third example embodiment.

FIG. 11 is a block diagram showing the hardware configuration of the earphone control device 20B in the third example embodiment.

FIG. 12 is a functional block diagram of the earphone control device 20B in the third example embodiment.

FIG. 13 is a block diagram showing the hardware configuration of the earphone 2C in the fourth example embodiment.

FIG. 14 is a block diagram showing the hardware configuration of the earphone control device 20C in the fourth example embodiment.

FIG. 15 is a diagram showing an example of the configuration of the reporting system S in the fifth example embodiment.

FIG. 16 is a flowchart showing the process flow of the reporting system S in the fifth example embodiment.

FIG. 17 is an example of the reporting information in the fifth example embodiment.

FIG. 18 is a functional block diagram of the earphone control device 20E in the sixth example embodiment.

FIG. 19 is a flowchart showing the process flow of the reporting system S in the sixth example embodiment.

FIG. 20 is a diagram showing the minimum configuration of the reporting system S in this disclosure.

FIG. 21 is a flowchart showing the flow of processing in the minimum configuration example embodiment of the reporting system S in this disclosure.

### EXAMPLE EMBODIMENT

Preferred example embodiments are described in detail below with reference to the accompanying drawings. The following example embodiments are not intended to limit the invention according to the claims, and not all of the combinations of features described in the example embodiments are essential to the invention. Two or more of the plurality of features described in the example embodiments may be arbitrarily combined. The same reference numbers are used for identical or similar configurations, and redundant explanations are omitted.

### First Example Embodiment

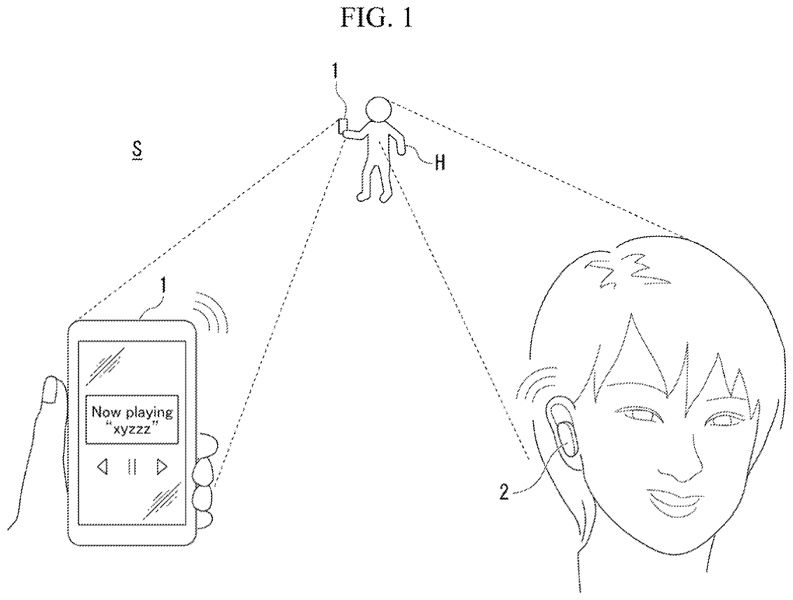

FIG. 1 shows an example of the configuration of a reporting system S in the first example embodiment. The reporting system S is equipped with an information communication device 1 and an earphone 2. A user H wears the earphone 2 in his or her ear. The user H is using the information communication device 1. The information communication device 1 is an electronic device provided with communication functions. The information communication device 1 can be, for example, a smartphone, a tablet computer, a wristwatch-type portable terminal, or a personal computer (PC).

In FIG. 1, music is being played by the information communication device 1, and the user H is listening to the music using the earphone 2. The information communication device 1 and the earphone 2 are connected to each other by wireless communication. Although not shown in the illustration, the earphone 2 may be connected to the information communication device 1 by a wire. The information communication device 1 and the earphone 2 may be configured as a single device, or yet another device may be included within the reporting system S. In this specification, “sound” such as sound waves and audio shall include non-audible sounds whose frequency or sound pressure level is outside the audible range.

The information communication device 1 is communicatively connected to the earphone 2. The information communication device 1 controls the operation of the earphone 2, transmits audio data for generating sound waves emitted from the earphone 2, and receives audio data obtained from the sound waves received by the earphone 2. Specifically, when the user H uses the earphone 2 to listen to music, information communication device 1 sends compressed data of music to the earphone 2.

When the earphone 2 is connected to a portable terminal for business use at an event site, hospital, or the like, the information communication device 1 may transmit audio data of business instructions to the earphone 2. In this case, the earphone 2 may transmit the audio data of the user H's speech to the information communication device 1. The information communication device 1 or earphone 2 may be equipped with the function of otoacoustic authentication using the sound waves received by the earphone 2. In the present specification, “sound” such as sound waves and audio shall include non-audible sounds whose frequency or sound pressure level is outside the audible range of humans.

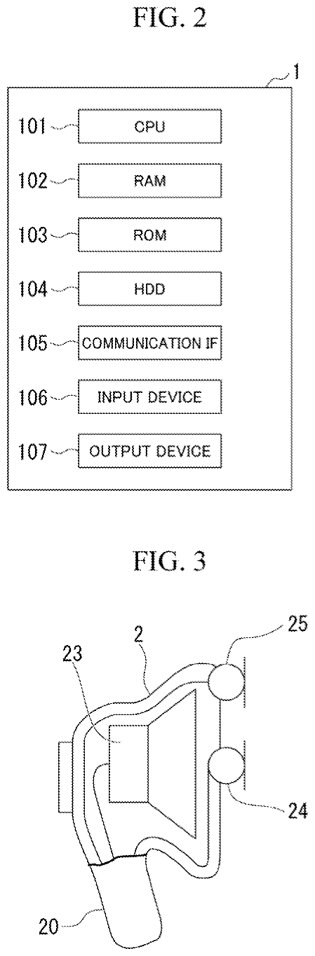

FIG. 2 is a block diagram showing the hardware configuration of the information communication device 1 in the first example embodiment. The information communication device 1 is provided with a central processing unit (CPU) 101, random access memory (RAM) 102, read-only memory (ROM) 103, and a hard disk drive (HDD) 104.

The information communication device 1 also has a communication interface (IF) 105, an input device 106, and an output device 107. The CPU 101, RAM 102, ROM 103, HDD 104, communication IF 105, input device 106, and output device 107 are interconnected via buses, wiring, and driving devices not shown in FIG. 2.

The CPU 101 performs predetermined calculations in accordance with programs stored in the ROM 103, HDD 104, and the like. The CPU 101 is a processing unit that also has the function of controlling each part of the information communication device 1. The RAM 102 consists of a volatile storage medium. The RAM 102 provides a temporary memory area. The temporary memory area is necessary for the operation of the CPU 101.

The ROM 103 consists of a nonvolatile storage medium. The ROM 103 stores necessary information such as programs used in the operation of the information communication device 1. The HDD 104 consists of a nonvolatile storage medium and is a storage device that temporarily stores data sent and received to/from the earphone 2, stores programs for operating the information communication device 1, and so on. The HDD 104 can be a solid-state drive (SSD) instead of a HDD. If the information communication device 1 is a smartphone or wristwatch-type portable terminal, the information communication device 1 does not have to be equipped with the HDD 104.

The communication IF 105 is a communication interface that enables transmission and reception of data and control information, and the like, to/from other devices. The communication IF 105 is based on standards such as Bluetooth (registered trademark) and Wi-Fi (registered trademark), for example.

The input device 106 is a device used by the user H to operate the information communication device 1. The input device 106 may be a touch screen panel, for example, or a keyboard, mouse, trackball, or pen input device.

The output device 107 is a graphical user interface (Graphical user interface:(GUI) and other display devices. The output device 107 is a liquid crystal display, organic electroluminescence (EL) display, and the like. The input device 106 and the output device 107 may be integrally formed as a touch panel.

The hardware configuration shown in FIG. 2 is an example. Additional devices other than those shown in FIG. 2 may be added, or some devices may not be provided. Some of the devices shown in FIG. 2 may be replaced by other devices with similar functions. Furthermore, some functions of this example embodiment may be provided by other devices via a network. The functions of this example embodiment may be distributed and realized in multiple devices. The hardware configuration shown in FIG. 2 can be modified as needed.

FIG. 3 is a block diagram showing the hardware configuration of the earphone 2 in the first example embodiment. The earphone 2 is equipped with earphone control device 20, a speaker 23, a microphone 24, and a proximity sensor 25.

The earphone 2 is an acoustic device that can be worn in the ear of the user H, and is typically a device capable of wireless communication such as a wireless earphone or wireless headset. The speaker 23 functions as a sound wave generator that emits sound waves toward the ear canal of the user H when worn, and is located on the wearing side of earphone 2. The microphone 24 is arranged on the wearing side of the earphone 2 so as to be capable of receiving sound waves that are reflected by the user H's ear canal, etc. when worn.

The proximity sensor 25 is an element that detects the degree of proximity or contact with an object. The proximity sensor 25 may be an infrared sensor. In this case, the proximity sensor 25 consists of a photodiode or similar device. The proximity sensor 25 functions as a wearing detection unit that detects infrared radiation emitted from the user H when wearing the earphone 2. The proximity sensor 25 is located on the wearing side of the earphone 2. The earphone control device 20 controls the speaker 23, microphone 24, and proximity sensor 25, and communicates with the information communication device 1.

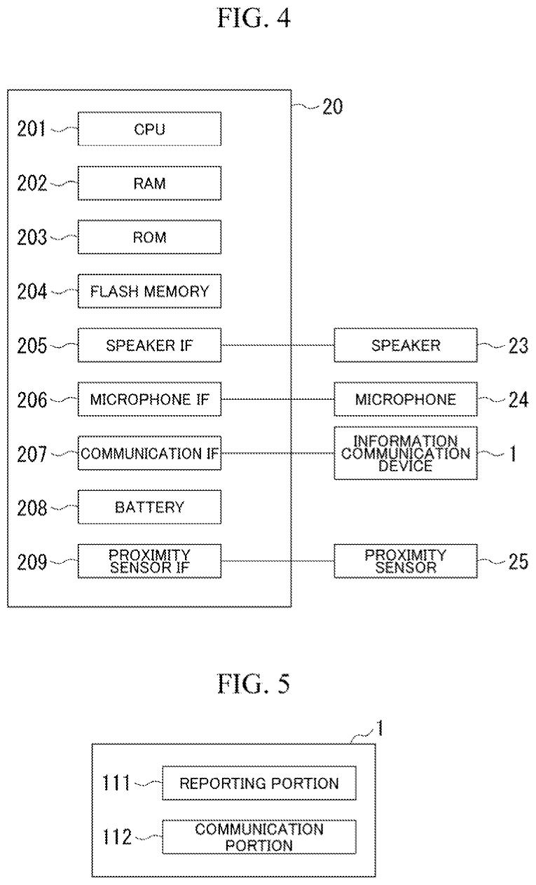

FIG. 4 is a block diagram showing the hardware configuration of the earphone control device 20 in the first example embodiment. The earphone control device 20 is provided with a CPU 201, RAM 202, ROM 203, flash memory 204, speaker IF 205, microphone IF 206, communication IF 207, battery 208, and proximity sensor IF 209. The various parts of the earphone control device 20 are interconnected via undisclosed buses, wiring, drive devices, and the like.

The CPU 201 is an operation device that performs predetermined operations according to a program stored in the ROM 203, the flash memory 204, and the like. The CPU 201 controls each portion of the earphone control device 20. The RAM 202 consists of a volatile storage medium. The RAM 202 provides the temporary memory area necessary for the operation of the CPU 201. The ROM 203 consists of a nonvolatile storage medium. The ROM 203 stores necessary information such as programs used in the operation of the earphone control device 20. The flash memory 204 is a storage device consisting of a nonvolatile storage medium. The flash memory 204 is used for temporary storage of data and storage programs for operation of the earphone control device 20.

The communication IF 207 is a communication interface that enables the transmission and reception of data and control information, etc., with other devices. The communication IF 207 is based on standards such as Bluetooth (registered trademark) and Wi-Fi (registered trademark), for example. When the earphone 2 is used in both ears of the user Has a pair, the two earphones 2 as a pair may be able to communicate with each other using the communication IF 207.

The speaker IF 205 is an interface for driving the speaker 23. The speaker IF 205 includes a digital-to-analog conversion circuit, amplifier, and other components. The speaker IF 205 converts audio data into analog signals and supplies them to the speaker 23. This causes the speaker 23 to emit sound waves based on the audio data.

The microphone IF 206 is an interface for acquiring signals from the microphone 24. The microphone IF 206 includes an analog-to-digital conversion circuit, an amplifier, and other components. The microphone IF 206 converts analog signals generated by sound waves received by the microphone 24 into digital signals. This allows the earphone control device 20 to acquire audio data based on the received sound waves.

The battery 208 provides the power necessary to operate the earphone 2. The battery 208 is, for example, a rechargeable battery. The battery 208 is built into the earphone 2, allowing the earphone 2 to operate wirelessly without a wired connection to an external power source.

The proximity sensor IF 209 is an interface for acquiring signals from the proximity sensor 25. The proximity sensor IF 209 includes an analog-to-digital conversion circuit, an amplifier, and the like. For example, if the proximity sensor 25 is an infrared sensor, the proximity sensor IF 209 converts the analog signal generated by the infrared light received by the proximity sensor 25 into a digital signal. The earphone control device 20 thereby obtains the detection data of the user H based on the received infrared light.

The hardware configuration shown in FIG. 4 is an example, and devices other than these may be added, or some of the hardware shown in FIG. 4 may not be provided.

Some of the devices may be replaced by other devices with similar functions. For example, the earphone 2 may be further equipped with an input device, such as a button, such that the input device can accept operations by the user H. The earphone 2 may be further equipped with a display, indicator light, or other display device to provide information to the user H. Thus, the hardware configuration shown in FIG. 2 can be modified as needed.

FIG. 5 is a functional block diagram of the information communication device 1 in the first example embodiment. The information communication device 1 is provided with a reporting portion 111 and a communication portion 112. The CPU 101 loads a program stored in the ROM 103, HDD 104, and the like into the RAM 102 to be executed. The reporting portion 111 may generate information to be reported based on the information acquired from the earphone 2 by the communication portion 112 with the communication IF 105. This allows the CPU 101 to perform the function of the reporting portion 111.

The communication IF 105 acquires notification information from the earphone 2 for the reporting portion 111 to report. The communication IF 105 may acquire the determination result by the determination portion 221 and/or the authentication result by the authentication portion 222 from the earphone 2. As a result, the communication IF 105 realizes the function of the communication portion 112. The operation of the determination portion 221 and the authentication portion 222 is described below.

The specific processes performed in the reporting portion 111 and the communication portion 112 are described below. These functions in the information communication device 1 may be realized by the earphone control device 20, or by the information communication device 1 and the earphone control device 20 working together.

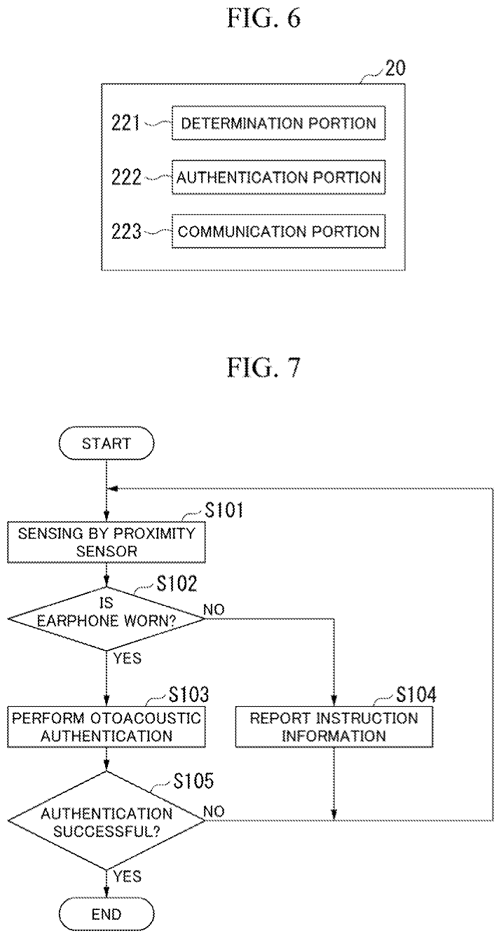

FIG. 6 is a functional block diagram of the earphone control device 20 in the first example embodiment. The earphone control device 20 is provided with a determination portion 221, an authentication portion 222, and a communication portion 223. The CPU 201 loads a program stored in the ROM 203, the flash memory 204 and the like into RAM 202 for execution. This allows the CPU 201 to perform the functions of the determination portion 221 and the authentication portion 222. The communication IF 207 outputs the results of the determination by the determination portion 221 and/or the authentication by the authentication portion 222 to the information communication device 1.

The CPU 201 may load a program stored in the ROM 203, flash memory 204 and the like into the RAM 202 for execution to generate information to be reported by the reporting portion 111 of the information communication device 1 based on the results of the determination by the determination portion 221 and/or the authentication by the authentication portion 222. In this case, the communication IF 207 outputs to the information communication device 1 the information reported by the reporting portion 111 of the information communication device 1.

The determination portion 221 determines whether or not the earphone 2 is properly worn by the user H. The authentication portion 222 performs authentication of the user H (authentication by otoacoustic authentication). The specific processes performed in the determination portion 221, the authentication portion 222, and the communication portion 223 are described below. These functions in the earphone 2 may be realized by the information communication device 1 or by the cooperation of the information communication device 1 and the earphone control device 20.

FIG. 7 is a flowchart showing the process flow of the reporting system S in the first example embodiment. In Step S101, the proximity sensor IF 209 measures the intensity of infrared radiation using the proximity sensor 25 (infrared sensor). The proximity sensor IF 209 advances the process to Step S102. In Step S102, the determination portion 221 uses the CPU 201 and at least one of the RAM 202, ROM 203, and flash memory 204, and the like to determine whether or not the user H is wearing the earphone 2 based on the intensity of the infrared radiation measured by the proximity sensor 25.

When the user H is wearing the earphone 2, the infrared radiation emitted from the user H is incident on the proximity sensor 25, so the intensity of the infrared radiation received by the proximity sensor 25 increases. Accordingly, the determination portion 221 can determine whether or not the device is worn based on a digital signal indicating the intensity of the infrared radiation received by the proximity sensor 25. For example, the determination criterion by the determination portion 221 may one for determining that the user H is wearing the earphone 2 when the intensity of the infrared radiation is equal to or greater than a threshold value. Alternatively, the determination criterion may be one for calculating a wear score based on the intensity of the infrared radiation or the change thereof, and determining that the user H is wearing the earphone 2 when the wear score is equal to or greater than a threshold value.

If the determination portion 221 determines in Step S102 that user H is wearing the earphone 2, the determination portion 221 proceeds to Step S103.

If the determination portion 221 determines in Step S102 that the user H is not wearing the earphone 2, the determination portion 221 generates instruction information that instructs the user to wear the earphone (earphone wearing instruction information). The communication portion 223 transmits the earphone wearing instruction information to the information communication device 1 by means of the communication IF 207. In this case, the determination portion 221 may generate information indicating that the user H is not wearing the earphone 2. The communication portion 223 may transmit the information indicating that the user H is not wearing the earphone 2 to the information communication device 1 by the communication IF 207. The determination portion 221 advances the process to Step S104.

In Step S103, the authentication portion 222 performs authentication of the user H using the CPU 201 and at least one of the RAM 202, ROM 203, flash memory 204, speaker IF 205 and speaker 23. In other words, the authentication portion 222 determines whether or not the user H is a registrant by means of otoacoustic authentication.

Otoacoustic authentication is a biometric authentication that collates the acoustic characteristics of the user H's ear canal. By emitting inspection sound waves into the ear canal of the user H by the speaker 23 and receiving the sound waves echoed by the user H's ear canal and the like by the microphone 24, the earphone 2 can acquire the acoustic characteristics of the user H's ear canal.

The authentication portion 222 can determine whether the user H is a registrant or not by collating the feature quantities extracted from the acquired acoustic characteristics of the ear canal with the feature quantities extracted from the acoustic characteristics of the registrant's ear canal. A registrant is a person who is an authorized user of the earphone 2 and for whom the acoustic characteristics or feature quantities of the ear canal have been registered. This authentication allows the earphone 2 functions to be restricted for unauthorized persons and prevents unauthorized persons from using the earphone 2.

In the present example embodiment, the acoustic characteristics of the registrant's ear canal are assumed to be stored in advance in the ROM 203 or the like, but the acoustic characteristics of the registrant's ear canal may be acquired from other devices such as the information communication device 1 during authentication.

The acoustic characteristics acquired in otoacoustic authentication are typically those resulting from resonance in the ear canal. Acoustic characteristics acquired in otoacoustic authentication can also include acoustic characteristics resulting from reverberation in tissues surrounding the ear canal, such as the skull. The examination sound waves do not have to be emitted directly into the ear canal. The examination sound waves may be emitted to a portion of the user H's head by a bone-conduction loudspeaker. After performing the otoacoustic authentication, the authentication portion 222 proceeds to Step S105.

In Step S104, the communication portion 112 acquires the earphone wearing instruction information from the earphone 2 by the communication IF 105. The reporting portion 111 outputs the earphone wearing instruction information from the output device 107. The reporting portion 111 may display the instruction information (earphone wearing instruction information) using a display device as the output device 107.

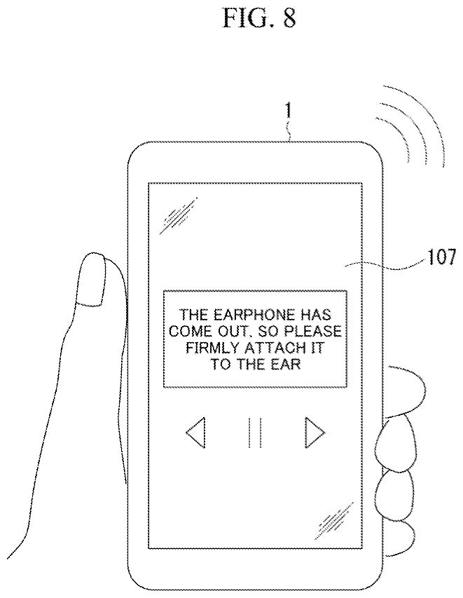

FIG. 8 shows an example of the instruction information in the first example embodiment. The reporting portion 111 displays “The earphone has come out, so please attach it firmly to your ear” on the display screen of the smartphone as the information communication device 1 (an example of a screen on the output device 107 or display element). The user may be informed by displaying a text message (character string) as the instruction information. The instruction information may be, for example, an image (still or moving) showing the action of putting the earphone that has been removed from the ear back in the ear.

In Step S104, the communication portion 112 may acquire information indicating that the user H is not wearing the earphone 2 from the earphone 2 by the communication IF 105. In this case, the reporting portion 111 may generate earphone wearing instruction information based on the CPU 101, at least one of the RAM 102, ROM 103 and HDD 104, and information indicating that the user H is not wearing the earphone 2. The reporting portion 111 may report the generated instruction information in the form shown in FIG. 8. The communication portion 112 may use the communication IF 105 to output an audio message to the earphone 2 prompting the wearing of the earphone 2. The reporting portion 111 advances the process to Step S101.

In Step S105, the authentication portion 222 determines whether or not the authentication of the user H by otoacoustic authentication is successful. If the authentication of the user H by otoacoustic authentication is successful, the authentication portion 222 ends the process. If the authentication of user H by otoacoustic authentication is not successful, the authentication portion 222 proceeds to Step S101.

As described above, the reporting system according to the first example embodiment has a determination means that determines whether a first earphone is worn in one ear of a user, a reporting means that, when the determination means determines that the earphone is not worn, reports instruction information instructing the user to wear the first earphone, and an authentication means that authenticates the user by otoacoustic authentication when the determination means determines that the first earphone is worn in the one ear after the reporting means has reported the instruction information. Otoacoustic authentication is an authentication method that sends out a test sound in the direction of the ear canal of one or the other ear of the user, and authenticates the user based on the reflected sound to the test sound.

This allows the system to prompt the user to wear the earphone if not worn by the user, and also allows for fast user authentication by otoacoustic identification.

The reporting means also reports by displaying instruction information as images or text on the screen of the display element.

This allows the user H to be notified of the instruction information in various formats, which enables effective prompting of the user H to wear the earphone 2.

### Second Example Embodiment

The reporting system S according to the second example embodiment of this disclosure is described with reference to the drawings. In the reporting system S of the second example embodiment, the configuration of the information communication device 1 and the earphone 2 is the same as in the first example embodiment.

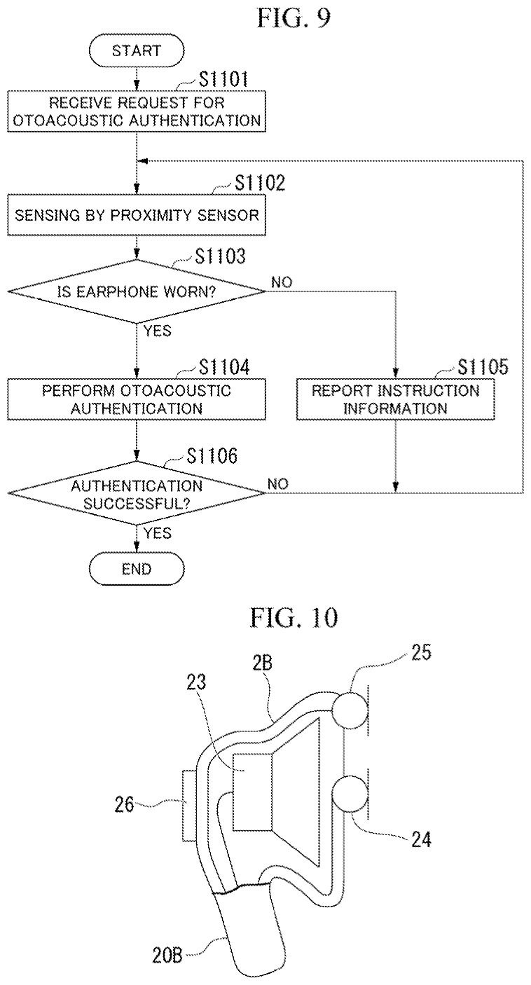

FIG. 9 is a flowchart showing the process flow of the reporting system S in the second example embodiment. At the beginning of the flowchart shown in FIG. 9, Step S1101 is added to the flowchart shown in FIG. 7. In the flowchart shown in FIG. 9, each process in Step S1102 to Step S1106 corresponds to each process in Step S101 to Step S105 in the flowchart shown in FIG. 7.

In Step S1101, the communication portion 223 receives the request for performing otoacoustic authentication via the communication IF 207. The request for performing otoacoustic authentication may be sent to the earphone 2 by the communication portion 112 of the information communication device 1 via the communication IF 207. The request for performing otoacoustic authentication may be input by the user H via the input device 106 of the information communication device 1. In this case, the input device 106 may be the touch screen panel of a smartphone serving as the information communication device 1.

The communication portion 223 then proceeds to Step S1102. Unlike the first example embodiment, processing after Step S1102 is not performed unless a request to perform otoacoustic authentication is received. The processing after Step S1102 is the same as the process after Step S101 in the first example embodiment.

As described above, the reporting system for the second example embodiment of this disclosure is further provided with a receiving means (communication portion 112 or communication portion 223) that receives a request to perform authentication of a user by otoacoustic authentication, and when the receiving means receives the request, the authentication means performs authentication of the user by otoacoustic authentication.

This minimizes the amount of processing in the execution of otoacoustic authentication, since otoacoustic authentication is performed only when an explicit request for the execution of otoacoustic authentication is received, in accordance with the wearing status of the earphone 2.

In Step S1101, a request for performing otoacoustic authentication may be received by the communication portion 112 of the information communication device 1 (e.g., smartphone) from another device and output by the communication portion 112 to the earphone 2, or may be input directly from the other device to the earphone 2 without going through the information communication device 1. In this case, the other device may be, for example, building access control equipment or an authentication device installed at airport baggage checkpoint gates, immigration gates, and the like.

The communication portion 223 may then output information indicating that the otoacoustic authentication was successful, either through the information communication device 1 or directly to the other device. The access control equipment or authentication device serving as the other device, upon receiving the information indicating that the otoacoustic authentication was successful, may allow the user H wearing the earphone 2 to enter or exit the building or authenticate the person as a registered user.

This allows for smooth user access control or authentication by other devices, in addition to minimizing the amount of processing required in performing otoacoustic authentication.

### Third Example Embodiment

The reporting system S according to the third example embodiment of this disclosure is described with reference to the drawings.

FIG. 10 is a block diagram showing the hardware configuration of earphone 2B in the third example embodiment. Unlike the first and second example embodiments, an earphone 2B is further equipped with an LED 26 on the outside of the earphone 2. The LED 26 should be located to the front of the side of the earphone 2. When the LED 26 is arranged on the earphone 2 so as to be located to the front of the side of the earphone 2 when the earphone 2 is worn by the user H, the light emitted by the LED 26 is easily recognized by the user H.

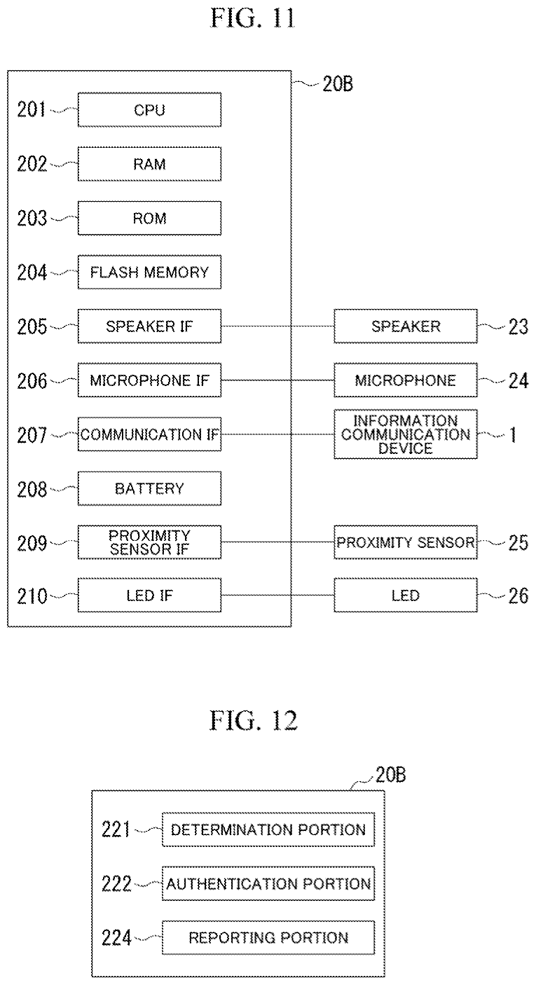

FIG. 11 is a block diagram showing the hardware configuration of the earphone control device 20B in the third example embodiment. Unlike the first and second example embodiments, the earphone 2B is further provided with a LED IF 210. The LED IF 210 is an interface for driving the LED 26.

FIG. 12 is a functional block diagram of the earphone control device 20B in the third example embodiment. Unlike the first and second example embodiments, the earphone 2B is provided with a reporting portion 224. In other words, in the present example embodiment, the entity that reports the earphone wearing instruction information is the earphone 2.

In the third example embodiment, the reporting portion 224 may report the instruction information by using either of the flowcharts shown in FIGS. 7 and 9. In Step S102 or Step S1103, the determination portion 221 may generate earphone wearing instruction information based on the CPU 201, at least one of the RAM 202, ROM 203 and flash memory 204, and information indicating that the user H is not wearing the earphone 2.

In Step S104 or Step S1105, the reporting portion 224 reports the generated instruction information by blinking the LED 26 using the LED IF 210.

As explained above, in the reporting system according to the third example embodiment of this disclosure, the reporting means reports instruction information by causing an LED (part of the earphone) to emit light.

Thereby it is possible to execute the reporting of the instruction information and the accompanying otoacoustic authentication process with only the earphone 2. In addition, the instruction information can be reported to the user H visually in a form that is easily recognized.

### Fourth Example Embodiment

The reporting system S according to the fourth example embodiment of this disclosure is described with reference to the drawings.

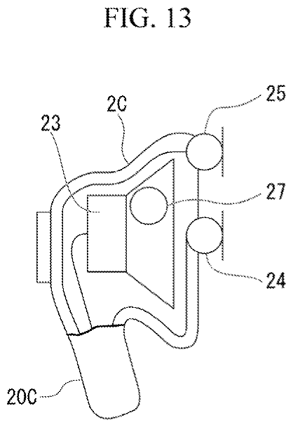

FIG. 13 is a block diagram showing the hardware configuration of earphone 2B in the fourth example embodiment. Unlike the first through third example embodiments, an earphone 2C has a vibrator 27 inside.

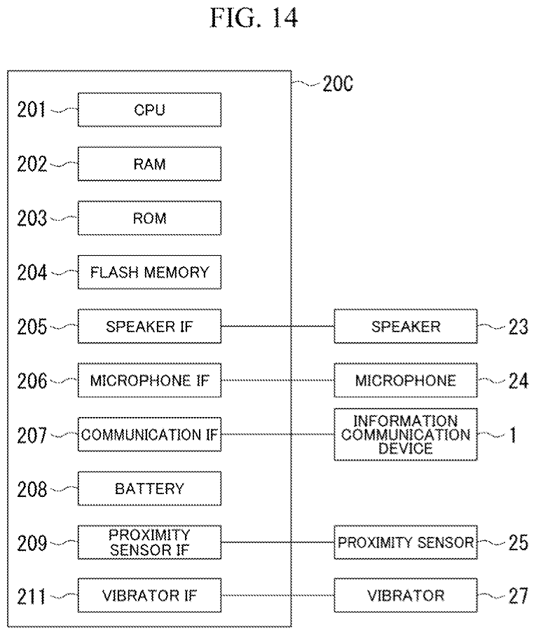

FIG. 14 is a block diagram showing the hardware configuration of an earphone control device 20C in the fourth example embodiment. Unlike the first through third example embodiments, an earphone 2C is further provided with the vibrator 27. A vibrator IF 211 is the interface for driving the vibrator 27.

The functional block diagram of the earphone control device 20C in the fourth example embodiment is similar to that of the earphone control device 20B in the third example embodiment, except for a LED IF 210 and the vibrator IF 211.

In the fourth example embodiment, the reporting portion 224 may report the instruction information by using either of the flowcharts shown in FIGS. 7 and 9. In Step S102 or Step S1103, the determination portion 221 may generate earphone wearing instruction information based on the CPU 101, at least one of the RAM 102, ROM 103 and HDD 104, and information indicating that the user H is not wearing the earphone 2.

In Step S104 or Step S1105, the reporting portion 224 reports the generated instruction information by vibrating the vibrator 27 using the vibrator IF 211.

As described above, in the reporting system according to the fourth example embodiment of the present disclosure, the reporting means reports instruction information by causing the vibrator inside the earphone 2 to vibrate.

Thereby it is possible to execute the reporting of the instruction information and the accompanying otoacoustic authentication process with only the earphone 2. In addition, the instruction information can be reported to the user H in an easily recognizable form using the vibration of the earphone 2 worn in the ear.

### Fifth Example Embodiment

The reporting system S according to the fifth example embodiment of this disclosure is described with reference to the drawings.

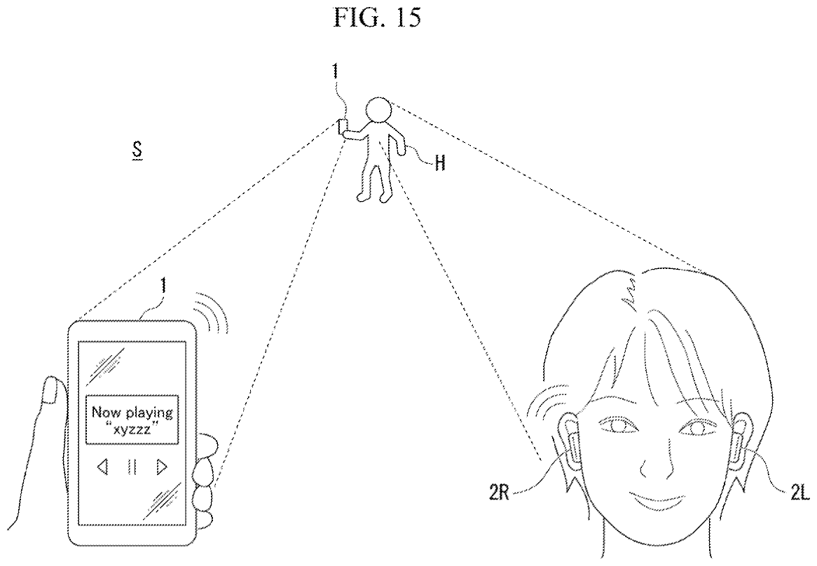

FIG. 15 shows an example of the configuration of a reporting system S in the fifth example embodiment.

In the present example embodiment, the user H uses an earphone 2R and an earphone 2L. The earphone 2R is used in the user H's right ear, and the earphone 2L is used in the user H's left ear.

The configuration of the earphones 2R and 2L can be any of the configurations in the first through fourth example embodiments. Either of the earphones 2R and 2L may communicate with the information communication device 1. The earphones 2R and 2L may communicate bidirectionally with each other by means of the communication IF 207. In the present example embodiment, the configuration of the information communication device 1 is described below taking the case of FIGS. 2 and 5 as examples, and the configuration of the earphones 2R and 2L are described below, taking the case of the configurations disclosed in FIGS. 3, 4, and 6 as examples.

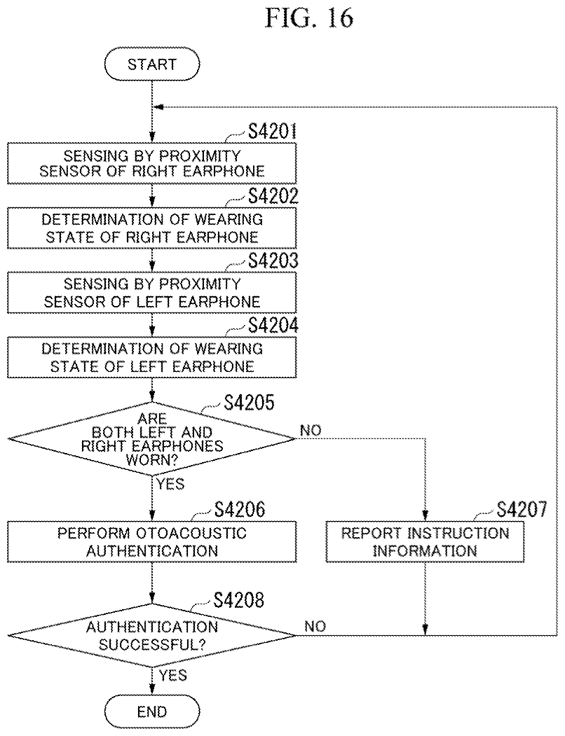

FIG. 16 is a flowchart showing the process flow of the reporting system S in the fifth example embodiment. In Step S4201, the proximity sensor IF 209R of the right earphone 2R measures the intensity of infrared radiation using the proximity sensor 25R (infrared sensor). The proximity sensor IF 209R advances the process to Step S4202.

In Step S4202, the determination portion 221R of the right earphone 2R determines whether the user H is wearing the earphone 2R based on the measured infrared radiation intensity. The determination portion 221R of the right earphone 2R generates information indicating whether or not the user H is wearing the right earphone 2R (right earphone 2R wearing state information). A communication portion 223R of the right earphone 2R transmits the right earphone 2R wearing state information to the information communication device 1 by a communication IF 207R.

In Step S4203, the proximity sensor IF 209L of the left earphone 2L measures the intensity of infrared radiation using the proximity sensor 25L (infrared sensor). The proximity sensor IF 209L advances the process to Step S4204. The sequence of steps S4201 and S4202 and the sequence of steps S4203 and S4204 may be interchanged in order.

In Step S4204, the determination portion 221L of the left earphone 2L determines whether the user H is wearing earphone 2L based on the measured infrared radiation intensity. The determination portion 221L of the left earphone 2L generates information indicating whether or not the user H is wearing the left earphone 2L (left earphone 2L wearing state information). A communication portion 223L of the left earphone 2L transmits the left earphone 2L wearing state information to the information communication device 1 by a communication IF 207L.

In Step S4205, the communication portion 112 acquires the right earphone 2R wearing state information and the left earphone 2L wearing status information by the communication IF 105. If the right earphone 2R wearing state information indicates that the right earphone 2R is worn by the user H, and the left earphone 2L wearing state information indicates that the left earphone 2L is worn by the user H, the reporting portion 111 generates information instructing the execution of otoacoustic authentication. The communication portion 112 uses the communication IF 105 to output information instructing the right earphone 2R and the left earphone 2L to execute the otoacoustic authentication. The communication portion 112 advances the process to Step S4206.

In Step S4205, if the right earphone 2R wearing state information indicates that the right earphone 2R is not worn by the user H and the left earphone 2L wearing state information indicates that the left earphone is not worn by the user H, the reporting portion 111 generates earphone wearing instruction information and outputs it from the output device 107. The reporting portion 111 may display instruction information instructing the wearing of the right earphone 2R and the left earphone 2L, using a display device as the output device 107.

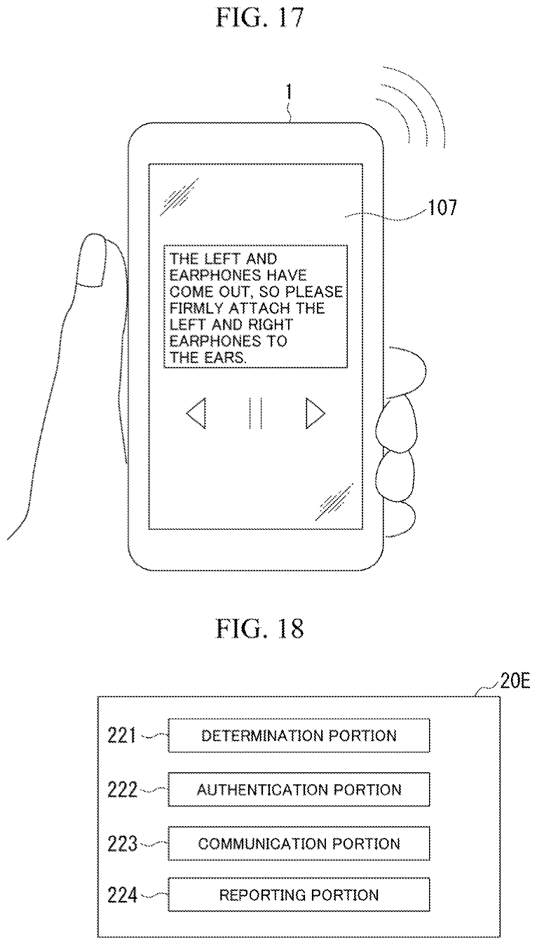

FIG. 17 is a diagram showing an example of the reporting information in the fifth example embodiment. As shown in FIG. 17, the reporting portion 111 may perform reporting by displaying the text message “The left and right earphones have been disconnected, please put the left and right earphones firmly in your ears,” as instruction information on the screen of the output device 107 (e.g., on the touch screen panel). The instruction information may be, for example, an image showing the action of putting an earphone that has become separated from the ear back in the ear.

In Step S4205, if the right earphone 2R wearing state information and left earphone 2L wearing state information indicate that only one of the right earphone 2R or left earphone 2L is not worn, the reporting portion 111 may transition to Step S4207. In Step S4207, the reporting portion 111 may display, using the display device as the output device 107, instruction information instructing the user to wear the earphone determined not to be worn, among the right earphone 2R and the left earphone 2L. In this case, the reporting portion 111 may report instruction information instructing the user to wear the earphone that has been determined not to be worn, in the manner shown in FIG. 8.

If only one of the right earphone 2R or left earphone 2L is not worn based on the right earphone 2R wearing status information and left earphone 2L wearing state information, the reporting portion 111 may generate wearing instruction information instructing the user to wear the earphone determined not to be worn.

The communication portion 112 may report the generated wearing instruction information to the earphone determined to be worn by using the communication IF 105. For example, if the right earphone 2R wearing state information indicates that the right earphone 2R is worn by the user H, and the left earphone 2L wearing state information indicates that the left earphone 2L is not worn by the user H, the reporting portion 111 may generate wearing instruction information to instruct the user to wear the left earphone 2L. The communication portion 112 may report the wearing instruction information instructing the user to wear the left earphone 2L to the right earphone 2R, by using the communication IF 105.

The communication portion 223R obtains the reported wearing instruction information via the communication IF 207R. Here, the speaker IF 205R may report to the user H the wearing instruction information that instructs the user to wear the left earphone 2L from the speaker 23R of the right earphone 2R in the form of sound or voice. The speaker IF 205R may output via voice from the speaker 23R the message “The left earphone is disconnected, so please put the left earphone firmly in your ear,” or may output some warning sound from the speaker 23R. When the reporting of instruction information is completed, the communication portion 223 advances the process to Step S4201.

The processing of Step S4206 and Step S4208 is similar to that of Step S103 and Step S105, respectively.

As described above, in the reporting system according to the fifth example embodiment of this disclosure, the determination means determines whether the second earphone (e.g., the left earphone 2L) is attached to the other ear (e.g., the left ear), and if the determination means has determined that the first earphone (e.g., the right earphone 2R) is not attached to one ear (e.g., the right ear) or the second earphone is not attached to the other ear, the reporting means reports information instructing the wearing of the earphone determined not to be worn, among the first earphone and the second earphone, by sound using the earphone determined to be worn among the first earphone and the second earphone.

Thereby since it is possible to use the earphone determined to be worn to report information instructing the wearing of the earphone determined not to be worn by sound or voice, it is possible to more easily prompt the user H to wear an earphone.

The reporting means may also report information instructing the wearing of the earphone determined not to be worn by light emission or illumination using the LED 26 of the earphone determined to be worn among the first earphone and second earphone.

The reporting means may also report information instructing the wearing of the earphone determined not to be worn with vibration by using the vibrator 27 of the earphone determined to be worn among the first earphone and second earphone.

Thereby since it is possible to use the earphone determined to be worn to report information instructing the wearing of the earphone determined not to be worn by illumination or vibration, it is possible to more easily prompt the user H to wear an earphone.

### Sixth Example Embodiment

The reporting system S according to the sixth example embodiment of this disclosure is described with reference to the drawings.

FIG. 18 is a functional block diagram of the earphone control device 20E in the sixth example embodiment. The earphone control device 20E is provided with the determination portion 221, the authentication portion 222, the communication portion 223, and the reporting portion 224 as functional blocks. The hardware configuration of the earphone 2 can be any of FIGS. 3, 10 and 13. The hardware configuration of the earphone control device 20E can be any of FIGS. 4, 11 and 14.

In the present example embodiment, the right earphone 2R and the left earphone 2L are provided with the communication portion 223R and the communication portion 223L, respectively. The right earphone 2R and the left earphone 2L communicate bidirectionally with each other using the communication portion 223R and the communication portion 223L. The right earphone 2R and the left earphone 2L may also communicate with the information communication device 1 using the communication portion 223R and the communication portion 223L.

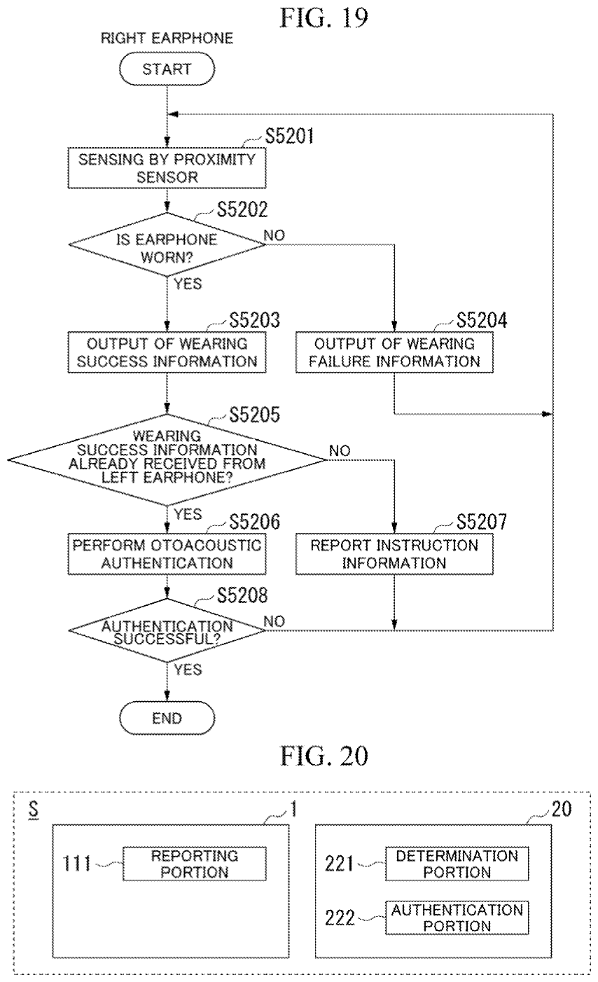

FIG. 19 is a flowchart showing the process flow of the reporting system S in the sixth example embodiment. FIG. 19 shows the process flow for the right earphone 2R, but the same process is performed for the left earphone 2L. In this case, with respect to the process flow of the left earphone 2L, the descriptions of the right earphone 2R and the left earphone 2L in the flowchart will be replaced.

In Step S5201, the proximity sensor IF 209R measures the intensity of infrared radiation using the proximity sensor 25R (infrared sensor). The right earphone 2R advances the processing to Step S5202.

In Step S5202, the determination portion 221R of the right earphone 2R determines whether the user H is wearing the earphone 2R based on the measured infrared radiation intensity. The determination portion 221R determines whether or not the user H is wearing the right earphone 2R. If the determination portion 221R determines that the user H is wearing the right earphone 2R, the determination portion 221R generates information indicating that the user H is wearing the earphone 2R (wearing success information). The determination portion 221R advances the processing to Step S5203.

If the determination portion 221R determines that the user H is not wearing the right earphone 2R, the determination portion 221R generates information indicating that the user H is not wearing the earphone 2R (wearing failure information). The determination portion 221R advances the processing to Step S5204.

In Step S5203, the communication portion 223R outputs the wearing success information to the left earphone 2L using the communication IF 207R. The communication portion 223R may use the communication IF 207R to output the wearing success information to the information communication device 1. The communication portion 223R advances the processing to Step S5205.

In Step S5204, the communication portion 223R outputs the wearing failure information to the left earphone 2L using the communication IF 207R. The communication portion 223R may use the communication IF 207R to output the wearing failure information to the information communication device 1. The communication portion 223R advances the processing to Step S5201.

In Step S5205, the determination portion 221R of the right earphone 2R determines whether or not it has already received the wearing success information from the left earphone 2L. If the determination portion 221R determines that the wearing success information has already been received from the left earphone 2L, the determination portion 221R advances the process to Step S5206. If the determination portion 221R determines that the wearing success information has been received from the left earphone 2L, the determination portion 221R advances the process to Step S5207.

The operation of Step S5206 and Step S5208 is similar to the processing of Step S103 and Step S105.

In Step S5207, the reporting portion 224R of the right earphone 2R reports wearing instruction information instructing the user to wear the left earphone 2L. In this case, the reporting portion 224R may report to the user H the audio of a message to the effect of prompting the wearing of the left earphone 2L by driving the speaker 23R with the speaker IF 205R, or may report to the user H by using a specific warning sound.

The reporting portion 224R may also report to the user H to the effect of prompting the wearing of the left earphone 2L by driving the LED 26R by the LED IF 210R to emit light in a specific pattern. The reporting portion 224R may also report to the user H to the effect of prompting the wearing of the left earphone 2L by driving the vibrator 27 with the vibrator IF 211 to vibrate in a specific pattern.

As described above, in the reporting system according to the sixth example embodiment of this disclosure, the determination means determines whether the second earphone (e.g., the left earphone 2L) is attached to the other ear (e.g., the left ear), and if the determination means has determined that the first earphone (e.g., the right earphone 2R) is not attached to one ear (e.g., the right ear) or the second earphone is not attached to the other ear, the reporting means reports information instructing the wearing of the earphone determined not to be worn, among the first earphone and the second earphone, by sound using the earphone determined to be worn among the first earphone and the second earphone.

Thereby since it is possible to use the earphone determined to be worn to report information instructing the wearing of the earphone determined not to be worn by sound or voice without using the information communication device 1, it is possible to more easily prompt the user H to wear an earphone.

Although the example embodiments of this invention have been described in detail with reference to the drawings, specific configurations are not limited to these example embodiments, but also include designs and the like to the extent that they do not depart from the gist of this invention.

In the example embodiment described above, the earphone 2 is shown as an example of a wearable device, but such a device is not limited to one worn in the ear as long as it can acquire the acoustic information necessary for processing. For example, a wearable device may be a bone-conducting acoustic device.

In the earphone 2 wearing determination by the proximity sensor 25, the proximity sensor 25 may be equipped with a light source such as an LED and a photodiode. The proximity sensor 25 may emit light from a light source and detect the reflected wave of that light with a photodiode. The determination portion 221 may determine the earphone 2 is being worn based on the presence or absence of the reflected wave of that light or the time until the reflected wave is received.

In the example embodiment described above, an infrared sensor is exemplified as a means of determining wearing by the proximity sensor 25, but it is not limited thereto provided determination of wearing is possible. For example, the proximity sensor 25 may emit sound waves into the ear canal of the user H, and the determination portion 221 may acquire the acoustic characteristics of the echo sound, such as the intensity of the echo sound and echo duration, and thereby make a wearing determination based on the acoustic characteristics of the echo sound. In this case, the speaker 23 and the microphone 24 function as devices not only for otoacoustic authentication, but also for determining the wearing of the device. Therefore, the device configuration can be simplified.

In the first example embodiment described above, it is assumed that the feature quantities are sent from the earphone 2 to the information communication device 1, but it is also possible to send data on the acoustic characteristics of the ear canal. In this case, the process of feature quantity extraction may be performed by the information communication device 1. In this example, the amount of communication increases, but the amount of computation within the earphone 2 can be reduced.

A processing method that records a program causing the operation of the configuration of the aforementioned example embodiments to realize the functions of the example embodiments in a storage medium, reads the program recorded in the storage medium as code and executes the code in a computer is also included within the scope of each example embodiment. In other words, a computer-readable storage medium is also included in the scope of each example embodiment. In addition, not only the storage medium in which the above-mentioned program is recorded, but also the program itself is included in each example embodiment. One or more components included in the example embodiments described above may be an ASIC (application specific integrated circuit), FPGA (field programmable gate array), or other circuit configured to realize the functions of each constituent element.

For example, a floppy (registered trademark) disk, hard disk, optical disk, optical magnetic disk, CD (Compact Disk)-ROM, magnetic tape, non-volatile memory card, and ROM can be used as the storage medium. The programs recorded on the storage media are not limited to those that execute processing by themselves, but also include those that operate on an OS (Operating System) and execute processing in collaboration with other software and expansion board functions.

The services realized by the functions of each of the above example embodiments can also be provided to users in the form of SaaS (Software as a Service).

The above example embodiments are merely examples of specific implementations in carrying out this disclosure, and the technical scope of this disclosure should not be interpreted as being limited thereto. In other words, this disclosure can be implemented in various forms without deviating from the technical concept thereof or the main features thereof.

FIG. 20 is a drawing showing the minimum configuration of the reporting system S in the present disclosure.

The reporting system S of the present example embodiment should at least be provided with the determination portion 221, the reporting portion 111, and the authentication portion 222.

The determination portion 221 determines whether or not the first earphone is worn in one of the user's ears.

The reporting portion 111 reports instruction information instructing the user to wear the first earphone when the determination portion 221 determines that the first earphone is not worn.

The authentication portion 222 performs authentication of the user by otoacoustic authentication if the determination portion 221 determines that the first earphone is worn in one ear after the reporting portion 111 reports the instruction information.

Otoacoustic authentication is an authentication method that sends out a test sound in the direction of the ear canal of one or the other ear of the user, and authenticates the user based on the reflected sound to the test sound.

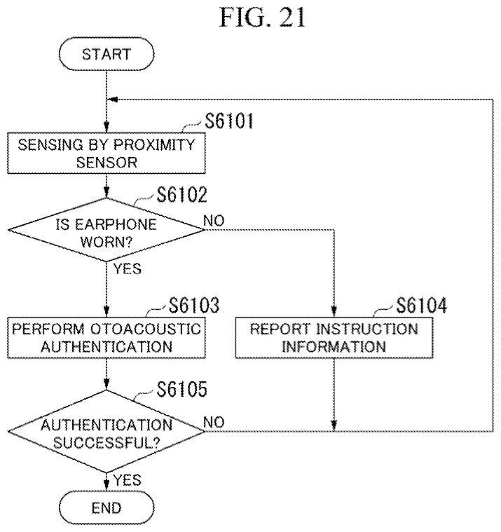

FIG. 21 is a flowchart showing the flow of processing in the minimum configuration example embodiment of the reporting system S in this disclosure.

The processing of Step S6101 is similar to the processing of Step S101. The determination portion 221 advances the process to Step S6102.

If the determination portion 221 determines in Step S6102 that the user H is wearing the earphone 2, the determination portion 221 proceeds to Step S6103. If the determination portion 221 determines that the user H is not wearing the earphone 2, the determination portion 221 generates instruction information that instructs the user to wear the earphone (earphone wearing instruction information). In this case, the determination portion 221 may generate information indicating that the user H is not wearing the earphone 2. The determination portion 221 advances the process to Step S6104.

The processing of Step S6103 is similar to the processing of Step S103. After performing the otoacoustic authentication, the authentication portion 222 proceeds to Step S6105.

In Step S6104, the reporting portion 111 outputs the earphone wearing instruction information from the output device 107. The reporting portion 111 may display the earphone wearing instruction information using a display device as the output device 107.

The reporting portion 111 may generate the earphone wearing instruction information based on the CPU 101, at least one of the RAM 102, ROM 103 and HDD 104, and information indicating that the user H is not wearing the earphone 2. The reporting portion 111 may report the generated instruction information in the form shown in FIG. 8. The reporting portion 111 may output an audio message to the earphone 2 prompting the wearing of the earphone 2. The reporting portion 111 advances the process to Step S6101.

The processing of Step S6105 is similar to the processing of Step S105.

This allows the system to prompt the user to wear the earphone if not worn by the user, and also allows for fast user authentication by otoacoustic identification.

Some or all of the above example embodiments may also be described as, but not limited to, the following Supplementary Notes.

### Supplementary Note 1

A reporting system including a determination means that determines whether a first earphone is worn in one ear of a user,
a reporting means that reports instruction information instructing the user to wear the first earphone when the determination means has determined that the first earphone is not worn, and
an authentication means that authenticates the user by otoacoustic authentication when the determination means has determined that the first earphone is worn in the one ear after the reporting means has reported the instruction information,
wherein the otoacoustic authentication is an authentication method that sends out a test sound in the direction of the ear canal of the one ear or other ear of the user, and authenticates the user based on the reflected sound to the test sound.

### Supplementary Note 2

The reporting system according to Supplementary Note 1, further comprising a receiving means that receives a request to authenticate the user by means of the otoacoustic authentication,
wherein the authentication means authenticates the user by means of the otoacoustic authentication when the receiving means has received the request.

### Supplementary Note 3

The reporting system according to Supplementary Note 1 or 2, wherein the reporting means reports by displaying the instruction information as an image or text on the screen of a display element.

### Supplementary Note 4

The reporting system according to Supplementary Note 1 or 2, wherein the reporting means reports the instruction information by making a part of the first earphone emit light.

### Supplementary Note 5

The reporting system according to Supplementary Note 1 or 2, wherein the reporting means reports the instruction information by making a part of the first earphone vibrate.

### Supplementary Note 6

The reporting system according to any one of supplementary notes 1 to 4, wherein the determination means determines whether a second earphone is worn in the other ear, and
when the determination means has determined that the first earphone is not worn in the one ear or the second earphone is not worn in the other ear,
the reporting means reports information instructing the wearing of the earphone that is determined to be not worn among the first earphone and the second earphone by sound using the earphone that is determined to be worn among the first earphone and the second earphone.

### Supplementary Note 7

A reporting method used for a computer, comprising:
a determination step that determines whether an earphone is worn in an ear of a user;
a reporting step that reports instruction information instructing the user to wear the earphone when it was determined in the determination step that the earphone is not worn; and
an authentication step that authenticates the user by otoacoustic authentication when it was determined in the determination step that the earphone is worn in the ear of the user, after the reporting of the instruction information in the reporting step,
wherein the otoacoustic authentication is an authentication method that sends out a test sound in the direction of the ear canal of an ear of the user, and authenticates the user based on the reflected sound to the test sound.

### Supplementary Note 8

A recording medium on which is stored a program that causes a computer to execute:
a determination step that determines whether an earphone is worn in an ear of a user;
a reporting step that reports instruction information instructing the user to wear the earphone when it was determined in the determination step that the earphone is not worn; and
an authentication step that authenticates the user by otoacoustic authentication when it was determined in the determination step that the earphone is worn in an ear of the user, after the reporting of the instruction information in the reporting step,
wherein the otoacoustic authentication is an authentication method that sends out a test sound in the direction of the ear canal of an ear of the user, and authenticates the user based on the reflected sound to the test sound.

### DESCRIPTION OF REFERENCE SIGNS

1 Information communication device
2 Earphone
101, 201 CPU
102, 202 RAM
103, 203 ROM
104 HDD
105, 207 Communication IF
106 Input device
107 Output device
23 Speaker
24 Microphone
25 Proximity sensor
26 LED
27 Vibrator
204 Flash memory
205 Speaker IF
206 Microphone IF
208 Battery
209 Proximity sensor IF
111, 224 Notification portion
112, 223 Communication portion
221 Determination portion
222 Authentication portion

What is claimed is:

1. A reporting system comprising:
at least one memory configured to store instructions; and
at least one processor configured to execute the instructions to:
determine whether a first earphone is worn in one ear of a user,
report instruction information instructing the user to wear the first earphone when it is determined that the first earphone is not worn, and
authenticate the user by otoacoustic authentication when it is determined that the first earphone is worn in the one ear after the instruction information is reported,
wherein the otoacoustic authentication is an authentication method that sends out a test sound in the direction of the ear canal of the one ear or other ear of the user, and authenticates the user based on the reflected sound to the test sound.

2. The reporting system according to claim 1, wherein the at least one processor is configured to execute the instructions to receives a request to authenticate the user by means of the otoacoustic authentication, and
authenticate the user by means of the otoacoustic authentication when the request is received.

3. The reporting system according to claim 1, wherein the at least one processor is configured to execute the instructions to report by displaying the instruction information as an image or text on the screen of a display element.

4. The reporting system according to claim 1, wherein the at least one processor is configured to execute the instructions to the instruction information by making a part of the first earphone emit light.

5. The reporting system according to claim 1, wherein the at least one processor is configured to execute the instructions to report the instruction information by making a part of the first earphone vibrate.

6. The reporting system according to claim 1, wherein the at least one processor is configured to execute the instructions to determine whether a second earphone is worn in the other ear, and
when it is determined that the first earphone is not worn in the one ear or the second earphone is not worn in the other ear, report information instructing the wearing of the earphone that is determined to be not worn among the first earphone and the second earphone by sound using the earphone that is determined to be worn among the first earphone and the second earphone.

7. A reporting method used for a computer, comprising:
determining whether an earphone is worn in an ear of a user;
reporting instruction information instructing the user to wear the earphone when it is determined that the earphone is not worn; and
authenticating the user by otoacoustic authentication when it is determined that the earphone is worn in the ear of the user, after the reporting of the instruction information,
wherein the otoacoustic authentication is an authentication method that sends out a test sound in the direction of the ear canal of an ear of the user, and authenticates the user based on the reflected sound to the test sound.

8. A non-transitory recording medium on which is stored a program that causes a computer to execute:
determining whether an earphone is worn in an ear of a user;
reporting instruction information instructing the user to wear the earphone when it is determined that the earphone is not worn; and
authenticating the user by otoacoustic authentication when it is determined that the earphone is worn in an ear of the user, after the reporting of the instruction information in the reporting step,
wherein the otoacoustic authentication is an authentication method that sends out a test sound in the direction of the ear canal of an ear of the user, and authenticates the user based on the reflected sound to the test sound.
